(19) United States
(12) Patent Application Publication
(10) Pub. No.: US 2007/0185437 A1
(43) Pub. Date: Aug. 9, 2007

(54) Needle-free syringe and the injection procedure

(76) Inventors: Boris Goldenberg, New York, NY (US); Ernest S. Geskin, (US)

Correspondence Address: Boris Goldenberg, 370 East 76 Street #C1008, New York, NY, 10021, US

(21) Appl. No.: 11/346,438

(22) Filed: Feb. 3, 2006

(51) Int. Cl. A61M 5/30

(52) U.S. Cl. 604/68

(57) ABSTRACT

The present invention relates to a needle-free syringe which has a closer that can be released or otherwise opened such that a greater percentage, and preferably all of the desired dose of fluid vaccine or other active agent is injected at a substantially uniform velocity. The dose is delivered by applying sufficient pressure to the fluid, which is stored in a suitable container, to push it toward a pressure-releasable closure such that the desired dose or volume of fluid is ejected from the device to the appropriate layer of the body.

### BACKGROUND OF THE INVENTION

Needle-free syringes administer a dose of fluid therapeutic agent or vaccine by ejecting a dose of the parental agent at the velocity exceeding the minimal level of safe and effective medicine delivery. Needle-free syringes have been well-known for more than fifty years. Conventionally, needle-free syringes consist of an enclosuer (container) which contains the dose of therapeutic agent, a piston movable inside the enclosure and an energy source that can drive a piston upon release, e.g., compressed gas or spring. When released, the stored energy accelerates the piston which expels the medicine at a speed sufficient for penetration into the patient's body. Existing needle-free syringes differ in the geometry, the shape and the size of the exit port and the design of the energy storage.

Needle-free syringes are currently being produced by a number of companies. They also have different patented designs. All patents can be split into three categories: 1—use a gas power for pressure of injection (for example, U.S. Pat. Nos. 6,210,359; 6,383,168B1; 6,645,170B2); 2—use a spring power for pressure of injection (for example, U.S. Pat. Nos. 5,722,953; 5,800,388; 6,123,684) and 3—use an electromechanical power and another mechanism for delivery of energy (for example, U.S. Pat. Nos. 6,939,323B2; 6,488,661B1).

Regardless of the syringe design, the amount of stored energy is reduced during the course of driving the piston and expulsion of the medicine. This reduction occurs regardless of the form of the storage and the exit port. As the pressure of the compressed gas drops or as the spring expands, there is a corresponding decrease in piston speed which results in the reduction of the rate of fluid exiting the syringe. In turn, this leads to a reduction of the speed and kinetic energy of the expelled medicine, and the speed of the trailing portion of the fluid ejected from the syringe is insufficient for penetration into the patient's body.

Thus, a part of the ejected fluid accumulates under the top layer of the skin, i.e., in the epidermis, which brings about unwanted consequences, including pain, redness and swelling at the site of injection. Variations, i.e., decrease, in the impact velocity of the medicine constitute a shortcoming of all existing needle-free syringes. It is an objective of this invention to prevent the reduction of the stream velocity of fluid medicines or vaccines expelled from a needle-free syringe, thereby prevent the accumulation of medicine under the skin.

The desired effect is attained by controlling the fluid pressure at the exit of the needle-free syringe. The minimal value of the exit pressure is attained by a pressure valve which closes the exit port when the fluid pressure drops below the critical level, e.g., after release of a volume of fluid from the syringe. Another control technique involves the use of two sources of energy injected sequentially into the fluid so that the second source is activated when the available energy of the first source drops below the critical level.

### SUMMARY OF THE INVENTION

This present invention utilizes two approaches for prevention of the deterioration of the fluid stream and thus assuring complete penetration of the ejected fluid. The minimal critical speed of the ejected fluid is assured by a pressure valve or functionally similar structure (hereinafter “flow regulator”) installed at the exit port of the syringe such that the valve is opened, i.e., fluid is expelled, only if the fluid pressure proximate to the pressure port exceeds the critical level. So the valve in this case also functions as a nozzle. If this pressure drops below the critical value, the flow regulator closes, and the fluid flow is interrupted. The flow regulator may be, e.g., an elastic (rubber) washer or formed of two sheets of plastic contacting each other at a closed angle at respective edges thereof.

The syringe body can also be used as a control valve. In this case the part of the syringe which forms an exit port is elastic and allows the port to be opened only at the fluid pressure exceeding the critical value. Another mode of maintaining the desired level of the velocity of the exiting fluid is the use of two or more sources of energy. When the amount of energy in a storage currently used for driving a piston drops, another source of energy is activated, and the energy rate of the supply to the fluid exceeds the critical level.

### BRIEF DESCRIPTION OF DRAWINGS

FIGS. 1A, 1B and 1C show a schematic of a needle-free syringe at three stages of injection where the outflow of the fluid is controlled by an elastic washer.

FIGS. 2A, 2B and 2C show a schematic of a needle-free syringe at three stages of injection where the fluid ejection is controlled by the elastic of the end or the valve of the end of the syringe body.

FIGS. 3A, 3B and 3C show a schematic of a needle-free syringe where the velocity of the ejected fluid is controlled by a dual energy supply to the fluid in the course of the ejection.

FIGS. 4A, 4B and 4C show a schematic of a needle-free syringe of the invention having a ball which applies additional force to the dual energy supply for ejecting the fluid.

### DETAILED DESCRIPTION OF THE INVENTION

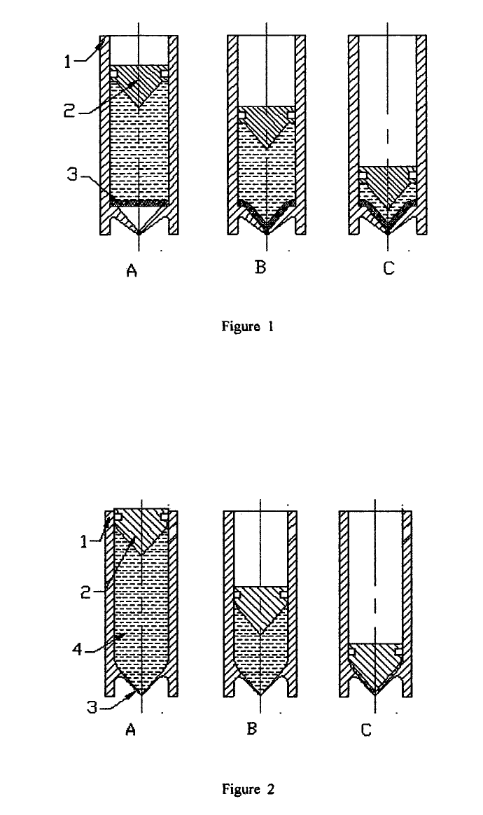

FIGS. 1A, B, C show three stages (A—before starting, B—middle position and C—end position) of a working needle-free syringe. Referring to FIG. 1A, the fluid 4 medicine or vaccine (or any other bioactive agent, e.g., vitamins or minerals) is contained in a container 1 made of plastic, glass or metal. The amount of the fluid accumulated in container 1 is determined by the position of piston or stopper or plunger 2 and flow regulator 3, in this case a rubber washer through which the fluid will flow under sufficient pressure or, alternatively, by physically contacting or piercing washer 3 with a pointed or conical end of stopper 2 or similar device.

Container 1 may be filled with fluid, and then stopper 2, which frictionally contacts the inner wall of container 1, may be inserted to contain the fluid. Thus, the end of stopper 2 that exerts pressure on the fluid 4 may be pointed, e.g., conical, or flat depending on the volume of fluid to be injected and whether or not it is necessary to pierce.

FIG. 1A shows the positions of the stopper 2 and washer 3 at rest. No external forces except for atmospheric pressure are exerted on stopper 2. Subsequently the pressure in the fluid beneath the piston is equal to the atmospheric, no expansion forces are applied to the washer 3 and the opening in washer 3 is closed.

FIG. 1B shows the middle step of pressure fluid 4 where the elastic washes 3 is deformed but the pressure is still not enough to open the piercing of the elastic washer.

Fluid is released when the stoper's motion deforms or expands washer 3 which is subjected to pressure equal to the pressure of the fluid inside container 1. When the expansion of washer 3 reaches the critical level, a puncture orifice is formed in washer 3 and opens, and the fluid dose begins exiting the container. The velocity of the exiting fluid is determined by the pressure developed by the fluid provided the pressure exceeds the critical level to force the washer open so that the fluid can escape. The require minimal fluid velocity is primarily determined by the elasticity of the material and the thickness of the washer 3. This feature assures additional stability of the washer position in the course of the fluid exit.

FIGS. 2A, B, C also show three stages (A—before starting, B—middle position and C—end position) of a working needle-free syringe, but the regulator of the pressure is located on the end of the container 1. So the end of the container 1 is made from a sterile plastic material which works the same way as the elastic washer that is described in FIG. 1, or a microvalve is installed in the end of the body of the syringe which also opens under the required pressure. Both the plastic end and the microvalve simultaneously function as nozzles.

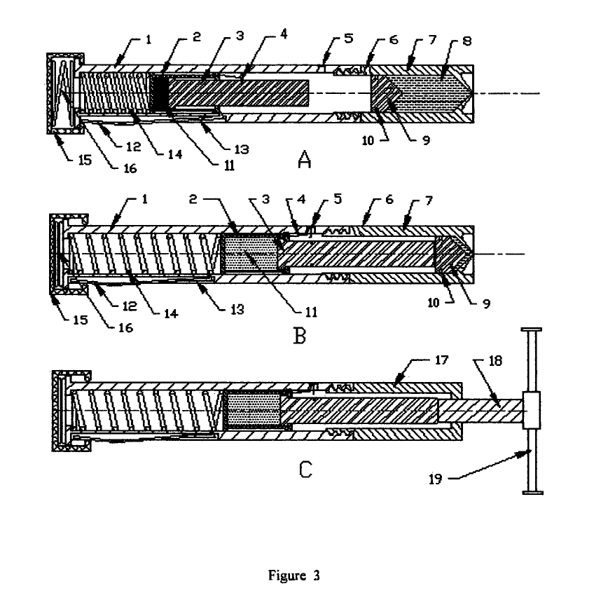

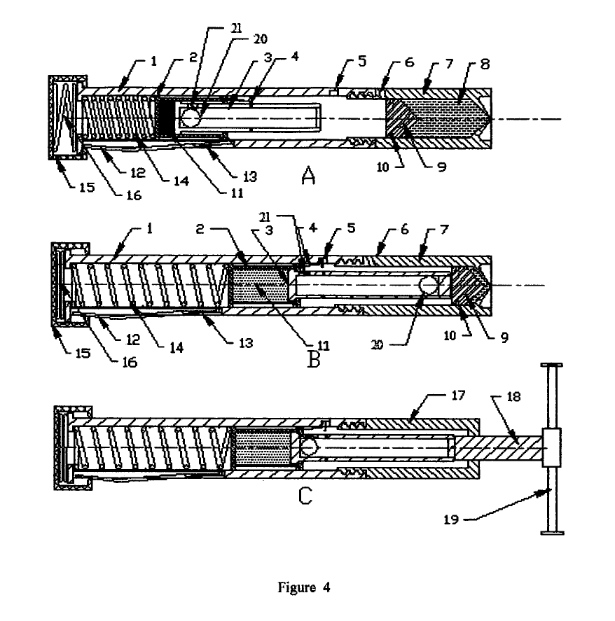

In FIGS. 3A, 3B, and 3C, a device utilizes two sources of energy to drive the piston 2, 3 and stopper 9. FIG. 3A, B, C (A—start position, B—energy of the spring and compressed gas release to drive the piston forward to apply pressure to the medicine, C—mechanism to return the energy to the start position). The syringe in this case actually contains the pistons: solid or hollow piston 3 which contacts the inside of hollow piston 2, which partially covers piston 3, which impacts piston (stopper) 9. A chamber 11 is formed between piston 3 and the inner wall of piston 2 and contains compressed gas. A spring 14 is provided to force piston 2 and piston 3 in a direction toward stopper 9 to compress the fluid. A spring 16 holds spring 14 in place by retention in button 15 of piston housing 1. Actuating mechanism 13 is provided to hold spring 14, piston 2 and piston 3 in start position until desired for use, and when activated by pushing the button 15 which pushes the lever 12 so that it no longer holds the pistons 2, 3 and the energy of spring 14 is released to drive the piston 2 with the piston 3 forward without releasing gas pressure yet.

Compressed gas is provided in chamber 11 to further provide force on the piston 3 to eject the fluid before there is a substantial drop in the energy being exerted on stopper 9 by piston 3. A retaining mechanism 4 having a lip is provided on the lateral portion of piston 2 to hold piston 3 in a position by contact with a receiving slot in the piston 3 so that the gas in chamber 11 remains compressed until the retaining mechanism is released once the piston reaches the piston shown in FIG. 3B. The retaining mechanism is released once because it is spring-like and it moves outward into receiving slots 5 of the inner wall of the housing, thereby allowing the compressed gas to exert force on the piston 3 propelling it further against the stopper 9. This dual-energy providing system allows a fairly rapid and continuous pressure to be exerted so that the fluid 8 is ejected at a substantially uniform pressure and velocity through the nozzle mechanism which is shown and described in FIG. 1 and FIG. 2. Thus, unlike with prior art devices, there is no or insubstantial slowing of fluid velocity toward the end of the spring or gas action because a second energy force replaces energy lost by the spring as it uncoils.

In operation, the pressure exerted on stopper 9 as well as the pressure exerted by the piston 2 on the fluid located beneath the piston increases. The stopper 9 with seal 10 is moved downward toward exit end of container 7 by force, e.g., by a spring and compressed air, or other means.

FIG. 3C shows the mechanism to return the energy to the start position. Instead of the container 7, the cylinder 17 is threaded onto the housing 1. The screw 18, rotated by lever 19, moves the piston 3, piston 2 and spring 14 back to the start position and simultaneously compresses the gas in chamber 11.

FIG. 4 shows a system that has three types of energy. Two types of energy and the mechanism to start them are the same as the system in FIG. 3. However, a ball 20 is fit in a hollow piston 3, which provides a third type of additional energy. Ball 20 is retained by ball holder 21 in the pre-injection position. When the device is activated, holder 21 releases ball 20, which provides additional force via inertia to drive piston 3 forward to deliver the dose of fluid. The ball may be used in conjunction with the spring and compressed gas, or may be used in conjunction with either of those.

The exit section of the device may simultaneously contain more than one exit port with regulators of pressure working as nozzles.

The exit section of the syringe that contacts the skin is made from soft elastic material for reducing the impact effect of the inertia the pistons.

What we claim is:

1. Needle-free syringe comprising:
a container, a piston and a driving mechanism for ejecting fluid medicine where the driving mechanism consists of more than one source of energy in one syringe according to a desired sequence.

2. Needle-free syringe according to the claim 1 where the additional sources of energy are engaged automatically when the effect of the previous source of energy decreases.

3. Needle-free syringe according to the claim 1 where one of source of energy is kinetic energy.

4. Needle-free syringe according to the claim 1 where additional sources of energy are the potential energy of a spring, pressure of gas and kinetic energy of a ball.

5. Needle-free syringe according to the claim 1 where the exiting section of the syringe contains a port opened when the fluid pressure exceeds a given level and closes when it drops below this level.

6. Needle-free syringe according to the claim 5 where the ejection of the fluid is stopped by a pressure elastic valve installed at the exit of the syringe and simultaneously is a nozzle of the syringe.

7. Needle-free syringe according to the claim 6 where the exit valve is an insertion of a punctured rubber washer where the puncture opens when the fluid pressure exceeds a given level and closes when it drops below this level.

8. Needle-free syringe according to the claim 1, 5, 6 where the exit section contains several exit holes equipped with the pressure valves.

9. Needle-free syringe according to the claim 1 where the end of the needle-free syringe is made from buffer elastic material.
